(19) United States
(12) Patent Application Publication
(10) Pub. No.: US 2008/0107648 A1
(43) Pub. Date: May 8, 2008

(54) Therapeutic methods for inhibiting tumor growth with DII4 antagonists

(75) Inventors: Irene Noguera, Bay Shore, NY (US); Gavin Thurston, White Plains, NY (US); Nicholas Gale, Yorktown Heights, NY (US); Eric Smith, New York, NY (US)

Correspondence Address: REGENERON PHARMACEUTICALS, INC, 777 OLD SAW MILL RIVER ROAD, TARRYTOWN, NY, 10591, US

(73) Assignee: Regeneron Pharmaceuticals, Inc.

(21) Appl. No.: 11/639,894

(22) Filed: Dec. 15, 2006

Related U.S. Application Data
(60) Provisional application No. 60/751,173, filed on Dec. 16, 2005.
(60) Provisional application No. 60/771,276, filed on Feb. 8, 2006.
(60) Provisional application No. 60/788,456, filed on Mar. 31, 2006.
(60) Provisional application No. 60/830,543, filed on Jul. 12, 2006.

(51) Int. Cl. A61K 39/395; A61P 43/00

(52) U.S. Cl. 424/133.1; 424/138.1; 424/178.1

(57) ABSTRACT

A therapeutic method for inhibiting tumor development or growth, comprising administering an agent capable of inhibiting human delta-like ligand 4 (DII4) activity to a subject in need thereof. In one embodiment, the agent is an anti-DII4 antibody or antibody fragment capable of inhibiting the binding of DII4 to a Notch receptor. In another embodiment, the agent is a fusion protein comprising the extracellular domain of DII4 or a fragment or variant thereof, fused to a multimerizing component such as an Fc domain. The method of the invention is useful for inhibiting tumor growth, particularly in tumors which are not responsive to other therapeutic agents.

### CROSS-REFERENCE TO RELATED APPLICATIONS

This application claims the benefit under 35 USC § 119(e) of U.S. Provisional application 60/751,173 filed 16 Dec. 2005, 60/771,276 filed 8 Feb. 2006, 60/788,456 filed 31 Mar. 2006, and 60/830,543 filed 12 Jul. 2006, which applications are herein specifically incorporated by reference in their entirety.

### BACKGROUND OF THE INVENTION

1. Field of the Invention

This invention relates to methods of inhibiting tumor growth with delta-like ligand 4 (DII4) antagonists. DII4 antagonist may be particularly useful for treating tumor growth in tumors which are unresponsive to other anti-tumor agents.

2. Description of Related Art

The Notch-signaling pathway is a system for cell-to-cell communication used by a wide range of eukaryotes for many biological processes, such as differentiation, proliferation, and homeostasis. Delta like 4 (DII4) or delta-like ligand 4 (DII4) (hereinafter “DII4”) is a member of the Delta family of Notch ligands which exhibits highly selective expression by vascular endothelium (Shutter et al. (2000) Genes Develop. 14:1313-1318). DII4 is a ligand for Notch receptors, including Notch1 and Notch 4. The nucleic acid and amino acid sequences for human and mouse DII4 are shown in SEQ ID NO:1-2 and SEQ ID NO:3-4, respectively. Gene targeted DII4 mice have been generated (Duarte et al. (2004) Genes & Dev. 18: doi: 10.1101/gad.1239004; Krebs et al. (2004) Genes & Dev. 18: doi: 10.1101/gad.1239204: Gale et al. (2004) Proc Natl Acad Sci USA 101:15949-15954).

### BRIEF SUMMARY OF THE INVENTION

The experiments described below show that DII4 antagonists are effective in inhibiting tumor growth, particularly in tumors which are not responsive to other anti-tumor therapeutics, such as a vascular endothelial growth factor (VEGF) antagonists.

In a first aspect, the invention features DII4 antagonists capable of inhibiting DII4. In one embodiment, the DII4 antagonist is an antibody or antibody fragment which specifically binds DII4 and blocks DII4 binding to a Notch receptor, such as for example, Notch1. In another embodiment, the DII4 antagonist of the invention is a fusion protein comprising the extracellular domain of DII4 or a fragment thereof fused to a multimerizing component.

The antibody or antibody fragment used in the method of the invention may be polyclonal or monoclonal, and may be humanized, chimeric, fully human. Preferably the antibody is a fully human monoclonal antibody or monoclonal antibody fragment. The antibody fragment may be, for example, a single chain antibody, an Fab, an F(ab′)₂, a peptibody, etc.

When the DII4 antagonist is a fusion protein, the extracellular domain of DII4 or a fragment or modified fragment thereof is fused to a multimerizing component. The multimerizing component is preferably an immunoglobulin domain, such as for example an Fc domain, e.g., a human Fc (SEQ ID NO:20). The fusion protein may optionally comprise a signal sequence, which may be native to the cell, recombinant, or synthetic.

In a second aspect, the invention features a pharmaceutical composition useful for inhibition of blood vessel growth or development, comprising an agent capable of inhibiting DII4 activity and a pharmaceutically acceptable carrier. In one embodiment, the agent is an antibody or antibody fragment which blocks the binding of DII4 to a Notch receptor. Preferably, the DII4 antagonist is a fully human antibody or fragment thereof capable of inhibiting the binding of DII4 to the Notch1 receptor. In another embodiment, the agent is a modified DII4 protein which is capable of binding its Notch receptor but such binding does not result in activation of the receptor.

In a third aspect, the invention features a method of treating a DII4-mediated condition, comprising administering an agent capable of inhibiting DII4 activity or expression. The DII4-mediated condition is a condition in which it is desirable to inhibit blood vessel growth or development. The DII4 antagonist of the invention may be particularly useful in treating tumors which are not responsive or are less than optimally responsive to other therapeutic agents. The DII4 antagonist may block production of functional blood vessels and oxygen delivery to the tumors. In specific embodiments, the antagonist is an anti-DII4 antibody or antibody fragment, or a fusion protein. The anti-DII4 antibody or antibody fragment preferably inhibits the binding of DII4 to the Notch1 receptor. The fusion protein of the invention comprises a fragment of the native extracellular region which retains the ability to bind to Notch receptors and lacks a transmembrane region and the cytoplasmic tail of DII4. In one embodiment, the DII4 antagonist of the invention is used therapeutically to treat tumors which are not responsive to treatment with a VEGF antagonist.

Other objects and advantages will become apparent from a review of the ensuing detailed description.

### BRIEF SUMMARY OF THE FIGURES

FIG. 1 shows that overexpression of DII4-Fc by C6 tumor cells results in smaller C6 tumors.

FIG. 2 shows that systemically-delivered DII4-Fc is highly effective in reducing HT1080 tumors relative to a receptor-based VEGF antagonist. Left hand panel: DII4-Fc or VEGF Trap protein given at time of tumor implant, tumors harvested day 25; Right hand panel: DII4-Fc or VEGF Trap protein given day 15 after implant, tumors harvested day 25.

FIG. 3 shows that purified DII4-Fc protein or polyclonal DII4 antibodies inhibits HT1080 tumor growth.

FIG. 4 shows inhibition of DII4 binding to the Notch1 receptor by the polyclonal antibodies to DII4, in a surface plasmon resonance (BiaCore®) assay.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

### DEFINITIONS

By the term “DII4-associated” or “DII4-mediated” condition or disease is meant a condition which is affected directly or indirectly by modulation of DII4 activity. More specifically, DII4 is now shown to be involved in blood vessel growth and development. Accordingly, in one embodiment, a DII4-associated condition treatable by the method of the invention is one in which it is desirable to inhibit or reduce DII4-mediated blood vessel growth or development or maturation, e.g., to inhibit tumor development.

By the term “inhibitor” or “antagonist” is meant a substance which retards or prevents a chemical or physiological reaction or response. Inhibition of DII4 activity may be direct, through inhibition of receptor activation with a blocking antibody, for example, or indirect, resulting from interference with expression of the gene encoding DII4. Common inhibitors include but are not limited to antisense molecules, antibodies, soluble receptors, antagonists and their derivatives, and modified DII4 ligands which bind their Notch receptor but are unable to activate signaling through such binding.

A “neutralizing” or “blocking” antibody, is intended to refer to an antibody whose binding to DII4 results in inhibition of the biological activity of DII4. This inhibition of the biological activity of DII4 can be assessed by measuring one or more indicators of DII4 biological activity. These indicators of DII4 biological activity can be assessed by one or more of several standard in vitro or in vivo assays known in the art (see examples below). Preferably, the ability of an antibody to neutralize DII4 activity is assessed by inhibition of DII4 binding to a Notch receptor, such as Notch1.

### General Description

The Delta-like/Notch signaling pathway is necessary to establish an organized and hierarchical vasculature during development. Targeted deletions of various Delta-like/Notch genes, including DII4, result in mice that die during embryonic development due to severe vascular defects. Using microarray analysis, we found that Delta-like ligand 4 (DII4) a VEGF-regulated gene in mouse xenograft tumor models. In addition, it was found that in these tumor models, DII4 expression was significantly higher in tumor vessels compared to those in adjacent normal skin. To explore the effects of blocking DII4/Notch signaling in tumors, xenograft studies were performed in mice, where a soluble DII4-Fc molecule was delivered locally by retrovirally mediated over-expression in tumor cells or was delivered systemically using an adenoviral approach or by injecting purified protein. All methods of delivering DII4-Fc resulted in reduced tumor growth compared to controls. Additionally, DII4-Fc treated tumor vessels were more highly branched than controls, forming fine networks with dense vascular sprouting, but these vessels were less efficient than in those of control tumors. As revealed by array and Taqman™ analysis, these effects were associated with a decrease in Notch signaling. Similar effects on tumor growth were also observed using a polyclonal antibody solution that was injected into mice systemically. This polyclonal antibody solution was also found to inhibit binding of DII4 to Notch1 receptor. Additionally, it was found that DII4-Fc is more effective at reducing the growth of certain tumors than a receptor-based blocker of VEGF (“VEGF trap”, U.S. Pat. No. 7,070,959). These findings show that DII4 plays a key role in tumor growth, and support DII4 as a target for the development of anti-angiogenic therapies.

### DII4 Antagonists

DII4 antagonists include antibodies to DII4 and fragments thereof capable of blocking the binding of DII4 to a Notch receptor (such as Notch1), fusion proteins comprising the extracellular domain of DII4 fused to a multimerizing component, or fragments thereof, and peptides and peptibodies (see for example, US patent publication 2003/0229023 Oliner et al. herein specifically incorporated by reference in its entirety).

DII4 antibodies. The term “immunoglobulin or antibody” as used herein refers to a mammalian, including human, polypeptide comprising a framework region from an immunoglobulin gene or fragments thereof that specifically binds and recognizes an antigen, which, in the case of the present invention, is a DII4 protein or portion thereof. If the intended antibody or antibody-like protein will be used as a mammalian therapeutic, immunoglobulin binding regions should be derived from the corresponding mammalian immunoglobulins. If the molecule is intended for non-therapeutic use, such as for diagnostics and ELISAs, the immunoglobulin binding regions may be derived from either human or non-human mammals, such as mice. The human immunoglobulin genes or gene fragments include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, IgG₃, IgG₄) as well as allotypes thereof.

An exemplary immunoglobulin (antibody) structural unit of human IgG, comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms antibody, as used herein, also includes antibody fragments either produced by the modification of whole antibodies, or those synthesized de novo using recombinant DNA methodologies (e.g., single chain Fv (scFv) single variable domains (Dabs)) or those identified using display libraries such as phage, E. coli or yeast display libraries (see, for example, McCafferty et al. (1990) Nature 348:552-554).

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). Antibodies that are isolated from organisms other than humans, such as mice, rats, rabbits, cows, can be made more human-like through chimerization or humanization.

“Humanized” or chimeric forms of non-human (e.g., murine) antibodies are immunoglobulins, immunoglobulin chains or fragments thereof (such as Fv, Fab, Fab′, F(ab′)₂ or other antigen-binding subsequences of antibodies) that contain minimal sequences required for antigen binding derived from non-human immunoglobulin. They have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. A typical chimeric antibody is thus a hybrid protein consisting of the V or antigen-binding domain from a mouse antibody and the C or effector domain from a human antibody. Humanized antibodies have variable region framework residues substantially from a human antibody (termed an acceptor antibody) and complementarity determining regions (CDR regions) substantially from a mouse antibody, (referred to as the donor immunoglobulin). See, Queen et al., Proc. Natl. Acad. Sci. USA 86:10029-10033 (1989) and WO 90/07861, U.S. Pat. Nos. 5,693,762, 5,693,761, 5,585,089, 5,530,101 and 5,225,539. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids. For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly; (2) is adjacent to a CDR region; (3) otherwise interacts with a CDR region (e.g. is within about 6 Å of a CDR region), or (4) participates in the V_{L}-V_{H} interface. Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

Methods for generating human antibodies include, for example, Velocimmune™ (Regeneron Pharmaceuticals), XenoMouse™ technology (Abgenix), the “minilocus” approach, and phage display. The Velocimmune™ technology (U.S. Pat. No. 6,596,541) encompasses a method of generating a high specificity fully human antibody to a select antigen. This technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody. In specific embodiment, the cell is a CHO cell.

The XenoMouse™ technology (Green et al. (1994) Nature Genetics 7:13-21) generates a mouse having both human variable and constant regions from both the heavy chain and kappa light chain loci. In an alternative approach, others have utilized a “minilocus” approach in which an exogenous Ig locus is mimicked through inclusion of individual genes from the Ig locus (see, for example, U.S. Pat. No. 5,545,807). The DNA encoding the variable regions can be isolated with or without being operably linked to the DNA encoding the human heavy and light chain constant region.

Alternatively, phage display or related display technologies can be used to identify antibodies, antibody fragments, such as variable domains, and heteromeric Fab fragments that specifically bind to DII4. (see for example US patent publication 2003/0229023).

Screening and selection of preferred immunoglobulins (antibodies) can be conducted by a variety of methods known to the art. Initial screening for the presence of monoclonal antibodies specific to DII4 may be conducted through the use of ELISA-based methods or phage display, for example. A secondary screen is preferably conducted to identify and select a desired monoclonal antibody. Secondary screening may be conducted with any suitable method known to the art. One preferred method, termed “Biosensor Modification-Assisted Profiling” (“BiaMAP”) is described in U.S. patent application publication 2004/101920, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody:antigen interactions. Alternatively, ELISA-based, bead-based, or Biacore®-based competition assays can be used to identify binding pairs that bind different epitopes of DII4 and thus are likely to cooperate to bind the ligand with high affinity.

DII4 fusion proteins. When the DII4 antagonist is a fusion protein, the multimerizing component may be selected from the group consisting of (i) an immunoglobulin domain, (ii) a truncated multimerizing component, (iii) an amino acid sequence between 1 to about 500 amino acids in length, optionally comprising at least one cysteine residue, (iv) a leucine zipper, (v) a helix loop motif and (vi) a coil-coil motif. In a preferred embodiment, the multimerizing component is an immunoglobulin domain, preferably an Fc domain, e.g., a human Fc (SEQ ID NO:20). The fusion protein may optionally comprise a signal sequence, which may comprise any sequence known to a skilled artisan for directing secretion of a polypeptide or protein from a cell, include natural or synthetic sequences. Generally, a signal sequence is placed at the beginning or amino-terminus of the fusion protein of the invention. Such a signal sequence may be native to the cell, recombinant, or synthetic. The components of the fusion protein of the invention may be connected directly to each other or connected via one or more spacer sequences. In one preferred embodiment, the components are fused directly to each other. In another preferred embodiment, the components are connected with a nucleic acid sequence encoding a spacer of 1-200 amino acids. Any spacer known to the art may be used to connect the protein components. A spacer sequence may also include a sequence used to enhance expression of the fusion protein, provide restriction sites, and allow component domains to form optimal tertiary and quaternary structures and/or to enhance the interaction of a component with its receptor. In one embodiment, the fusion protein of the invention comprises one or more peptide sequences between one or more components that is (are) between 1-25 amino acids.

The extracellular domain of DII4 is composed of a Delta/Serrate/Lag-2 (DSL) domain and a tandem of eight epidermal growth factor (EGF)-like repeats. Generally, the EGF domains are recognized as occurring at about position 218-251 (domain 1), 252-282 (domain 2), 284-322 (domain 3), 324-360 (domain 4), and 362-400 (domain 5), with the DSL domain at about position 173-217 and the N-terminal domain at about position 27-172 of hDII4 (SEQ ID NO:2). In specific embodiments, the hDII4 antagonist capable of inhibiting DII4 activity is DSL-hFc comprising about amino acid 27 to about 172 of SEQ ID NO:2 fused to hFc (SEQ ID NO:20) (SEQ ID NO:21), N-terminal domain-DSL-hFc comprising about 27-217 of SEQ ID NO:2 fused to hFc (SEQ ID NO:22), EGF domains 1-5-hFc comprising about 218-400 fused to hFc (SEQ ID NO:23), EGF domains 1-4-hFc comprising about 218-360 fused to hFc (SEQ ID NO:24), EGF domains 1-3-hFc comprising about 218-322 fused to hFc (SEQ ID NO:25), EGF domains 1-2-hFc comprising about 218-282 fused to hFc (SEQ ID NO:26), or variants thereof optionally comprising linkers between the domain components. The components of the fusion protein may also be arranged in a variety of configurations while retaining the ability to act as DII4 antagonists.

### Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

### Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a DII4-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 0.5 to 20 milligrams of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

### Combination Therapies

In numerous embodiments, the DII4 antagonists of the present invention may be administered in combination with one or more additional compounds or therapies. For example, multiple fusion proteins or anti-DII4 antibodies can be co-administered, or be administered in conjunction with one or more therapeutic compounds. In a preferred embodiment, the DII4 inhibitor of the invention is administered with a VEGF antagonist, such as an anti-VEGF antibody or a VEGF trap. Preferred embodiments of a VEGF trap (as described in WO 00/75319, which publication is herein specifically incorporated by reference in its entirety) is VEGFR1R2-FcΔC1 (a).

The term “cytotoxic agent” as used herein refers to a substance that inhibits or prevents the function of cells and/or causes destruction of cells. The term is intended to include radioactive isotopes (e.g. I¹³¹, I¹²⁵, Y⁹⁰ and Re¹⁸⁶), chemotherapeutic agents, and toxins such as enzymatically active toxins of bacterial, fungal, plant or animal origin, or fragments thereof.

Combination therapy includes administration of a single pharmaceutical dosage formulation which contains a DII4 antagonist of the invention and one or more VEGF antagonist(s); as well as administration of a DII4 antagonist and one or more additional agent(s) in its own separate pharmaceutical dosage formulation. For example, a DII4 antagonist and a cytotoxic agent, a chemotherapeutic agent or a growth inhibitory agent can be administered to the patient together in a single dosage composition such as a combined formulation, or each agent can be administered in a separate dosage formulation. Where separate dosage formulations are used, the fusion protein of the invention and one or more additional agents can be administered concurrently, or at separately staggered times, i.e., sequentially.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

A “growth inhibitory agent” when used herein refers to a compound or composition which inhibits growth of a cell, especially a cancer cell either in vitro or in vivo. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Targeting the DII4 Gene in Mice

Gene Targeting. Velocigene™ technology (Valenzuela et al. (2003) Nat. Biotechnol. 21:652-9) was used to generate a precise deletion and exchange of the DII4 coding region, extending from the initiation to the termination codon (corresponding an 8.1 kB region comprising all of the coding exons and intervening introns), with the b-galactosidase reporter gene as well as a neomycin selection cassette. Briefly, a bacterial artificial chromosome (BAC) containing the 8.1 kb DII4 coding region and 140 Kb of flanking sequences (clone 475d4 from a 129/SvJ BAC library obtained from Incyte Genomics) was modified to generate a BAC-based targeting vector which was then linearized and used as a targeting vector to replace the DII4 gene in F1H4 (C57BU6:129 hybrid) mouse embryonic stem (ES) cells. Correctly targeted embryonic stem cells were identified using the loss of native allele (LONA) assay (Valenzuela et al. (2003) supra). Two independent correctly targeted ES lines were used to generate chimeric male mice that were complete transmitters of ES-derived sperm. Chimeras were then bred to C57BL/6 and/or ICR females to generate F1 mice or embryos, which were genotyped by LONA assays and β-galactosidase histochemical assays. Mice derived from both ES lines behaved identically, and pooled data from both clones were used for statistics.

Results. Targeting the DII4 gene in mice resulted in embryonic lethality and severe vascular defects, even in mice targeted at a single allele (see Gale et al. (2004) Proc Natl Acad Sci USA 101:15949-15954).

Tumor implantations. Lewis lung carcinoma cells (ATCC) were subcutaneously implanted into the flank of DII4 chimeric mice, harvested after 16 days, cut into 80 micron sections, and stained for CD31/PECAM or β-galactosidase as described (Holash et al. (2002) Proc Natl. Acad. Sci. USA 99:11393-8).

PECAM and reporter staining. Staining of whole-mounted embryos, as well as tissue sections from embryos and adults, were performed as previously described for CD31/PECAM to define the vascular endothelium and for β-galactosidase to visualize the DII4 reporter gene product (Gale et al. (2004) PNAS 101:15949-54).

### Example 2

### DII4-Fc Construct and Mouse Xenograph Studies

DII4-Fc (-TM) construct. A nucleic acid sequence was constructed having 2297 nucleotides corresponding to the extracellular domain of human DII4 (SEQ ID NO:1) without the transmembrane (-TM) domain, with a human Fc domain. The encoded amino acid sequence had 765 amino acids of DII4 protein (SEQ ID NO:18) and a molecular weight of approximately 85 kDa.

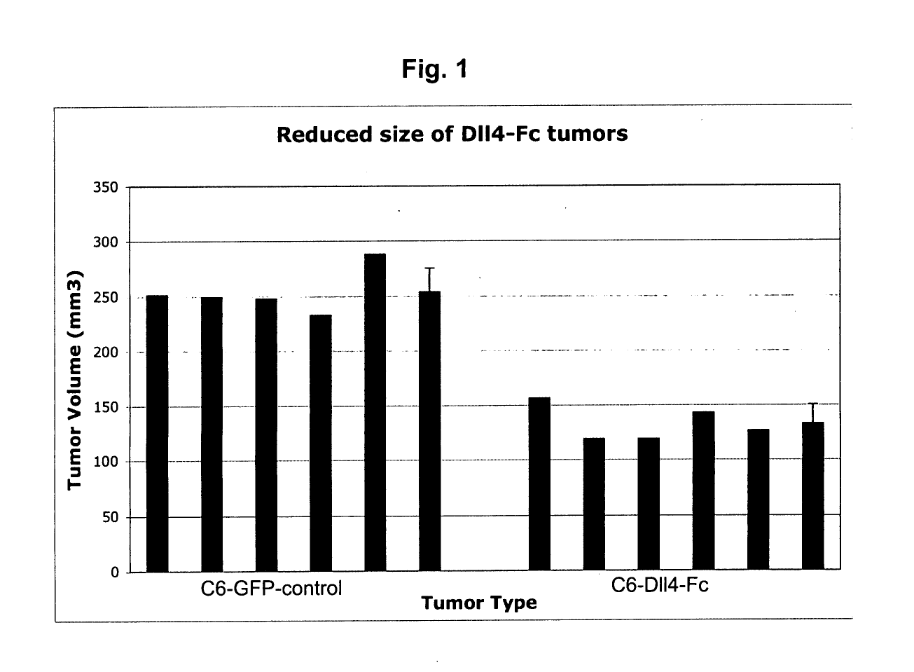

FIG. 1 shows that DII4-Fc over-expression by C6 tumor cells resulted in smaller C6 tumors (mean ±SD).

Retroviral engineering of tumor cells to over-express DII4-Fc. C6 rat glioma tumor cells (ATCC) were infected with retrovirus to over-express green fluorescent protein (GFP) and soluble DII4-Fc; cells infected with GFP alone were used as controls. Cells were FACS sorted for GFP fluorescence twice.

Retrovirus delivered DII4-Fc. 10⁶ cells/mouse were implanted subcutaneously into the shaved right flank of male SCID/CB17 mice (8-10 wk old) with either GFP or DII4-Fc retrovirally engineered C6 cells.

Tumor volume measurements: After tumors became palpable, size measurements were recorded every three days using a caliper (size=(length×width²)/2). Once animals were sacrificed, ex vivo measurements were obtained with calipers and volume was calculated using the formula length×width×height).

Tumor Histology. Twelve to sixteen days after tumor cell implantation, tumors were harvested and processed for histological or expression analysis. Tumors were cut into 80 μm sections, stained with antibodies to CD31/Pecam-1 followed by DAB-peroxidase reaction, and counterstained with pyronin Y. Vessel morphometric analysis was performed using the NIH Image 1.62 analysis program.

Northern Blotting and Real Time-PCR. Total RNA was prepared from tumor tissue using Trizol reagent (Life Technologies, Grand Island, N.Y.). RNA (10 mg) was separated on 1.2% agarose gels, transferred to nylon membrane and immobilized by UV crosslinking. After prehybridization, 32P-labeled DII4 or glyceraldehyde-3-phosphate dehydrogenase (GAPDH)-specific probes were added, and the filters were hybridized at 42° C. overnight. Stringent washes were performed by standard protocols (one wash 0.5×SSPE buffer followed by two washes with 0.2×SSPE buffer performed at 55° C. for 30 minutes each). An autoradiograph was obtained after 48 h exposure to x-ray film with intensifying screens. In addition, tissue specific expression was analyzed in separate reactions using the Taqman® (Applied Biosystems, Foster City, Calif.) real-time PCR chemistry and detection system with the primers pairs and labeled probes specific for DII4, the notch receptors 1 and 4 and notch downstream targets, Hes1, Hey2, HeyL and Nrarp. The number of cycles necessary to reach the threshold for amplification of the cDNA (or CT values) was obtained, and normalized to a housekeeping reference (GAPDH) (=2-DCT). The results were normalized to a baseline, the vehicle control for the experiment, giving the relative mRNA abundance change (=2DDCT) and is expressed as the mean ±s.e.m. for at least 4 separate samples run in triplicate (Livak and Schmittgen (2001) Methods. December; 25(4):402-8).

Quantitative RT-PCR analysis for DII4, HeyL, Nrarp and Hes1. The RT-PCR analysis was performed as described (Livak et al. (2001) Methods 24:402-8). Results are expressed as the ratio of the amount of the RNA of interest to the amount of control RNA (GAPDH) as described (Daly et al. (2004) Genes Dev. 18:1060-71) on an Applied Biosystems 7900HT using specific primers and probes as follows: DII4 Primers: DII4-1574F (SEQ ID NO:9) and DII4-1644R (SEQ ID NO:10) and DII4 Probe: DII4-1594T (SEQ ID NO:11); HeyL Primers: mHeyL-135F (SEQ ID NO:12) and mHeyL-216R (SEQ ID NO:13) and HeyLProbe: mHeyL-154T (SEQ ID NO:14); Nrarp Primers: mNrarp-350F (SEQ ID NO: 15) and mNrarp-418R (SEQ ID NO:16) and Nrarp probe: mNrarp-373T: (SEQ ID NO:17) and mHes1 (ID Mm00468601 ml, Hes1) (ABI, Assay on demand services). cDNAs were derived from C6-DII4-Fc and C6-DII4 tumors

In vitro assay to determine if secreted DII4-Fc expressed in C6 cells can activate Notch signaling in HUVEC. 4×10⁵ HUVEC cells were plated onto 60 mm dish to obtain ˜50% confluent cultures the following day. The next day, 8×10⁵C6 cells were plated on top of HUVECs. After 24 hrs of co-culture, cells were scrapped into 1 ml of Tri Reagent and total RNA was prepared as previously described. Samples were analyzed by Taqman® using human specific Hes1, HeyL and Nrarp probes.

### Example 3

### Effect of Systemic Administration of DII4-Fc

DII4-Fc protein. Plasmid encoding DII4-Fc cDNA construct described above was transfected into CHO cells, and secreted protein was purified from the supernatant. DII4-Fc protein was purified and used to treat tumor bearing mice via subcutaneous injection (10 mg/kg, 3× per week).

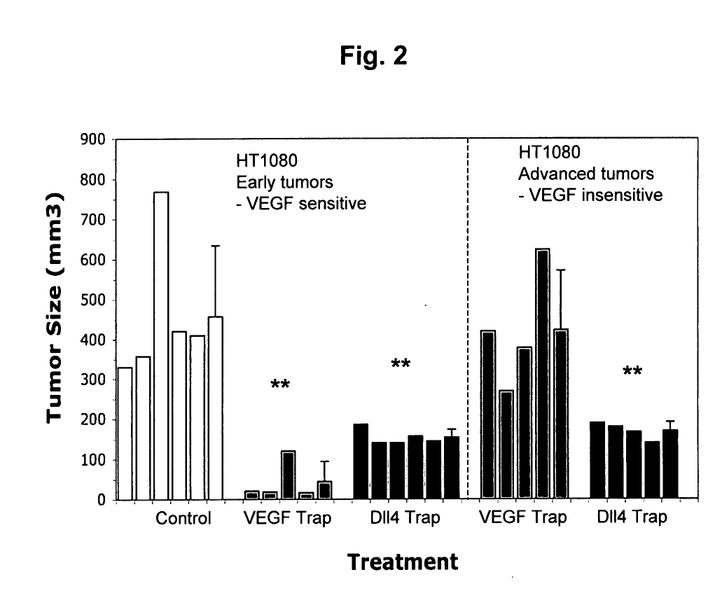

Results. Experiments were conducted in which HT1080 tumors were implanted into mice as described above at day 0, Starting on either day 0 or day 15 (at 100 mm³ in size), mice were treated with purified DII4-Fc protein (10 mg/kg, 3× per wk) or control protein. Other groups were treated with VEGF antagonist (VEGF Trap, SEQ ID NO:19) at a dose of 25 mg/kg, three times per week. The results are shown in FIG. 2. In tumors treated from day 0 (left hand side), both VEGF antagonist and DII4-Fc were effective at controlling tumor growth. In tumors treated from 100 mm³ in size (right hand side), DII4-Fc was again effective at controlling tumor growth, and was in fact more effective than VEGF antagonist.

Quantification of circulating DII4-Fc and hFc. Serum samples obtained from GFP or DII4-Fc treated mice bearing tumors were analyzed by ELISA assay. ELISA was performed by coating plates with hFc as the capture antibody, blocked with 0.2% I-Block solution (Tropix) and using hFc conjugated to peroxidase as a report antibody. Purified hFc and DII4-Fc proteins were included as standard curves.

VEGF-inhibitor treatment. VEGF trap (R1R2) (Regeneron Pharmaceuticals) (SEQ ID NO:19) or placebo (5% vol/vol PBS/glycerol) was administered subcutaneously to mice bearing 100 mm3 tumors at a dose of 25 mg/kg every three days until the end of the study.

Adenovirus delivery of DII4-Fc. Other experiments not shown have used adenovirus to deliver DII4-Fc systemically. C6, HT1080, or MMT tumor cells were implanted subcutaneously into the shaved right flank of male SCID/CB17 mice (8-10 wk old). After 24 hours, 1×10⁹ pfu of adeno-hFc or adeno-DII4-Fc was injected into the jugular vein of the mice. Similar results on tumor growth were seen with adeno-DII4-Fc as with systemic treatment with DII4-Fc protein.

### Example 4

### Effect of Polyclonal Antibodies to DII4-Fc on HT1080 Tumors

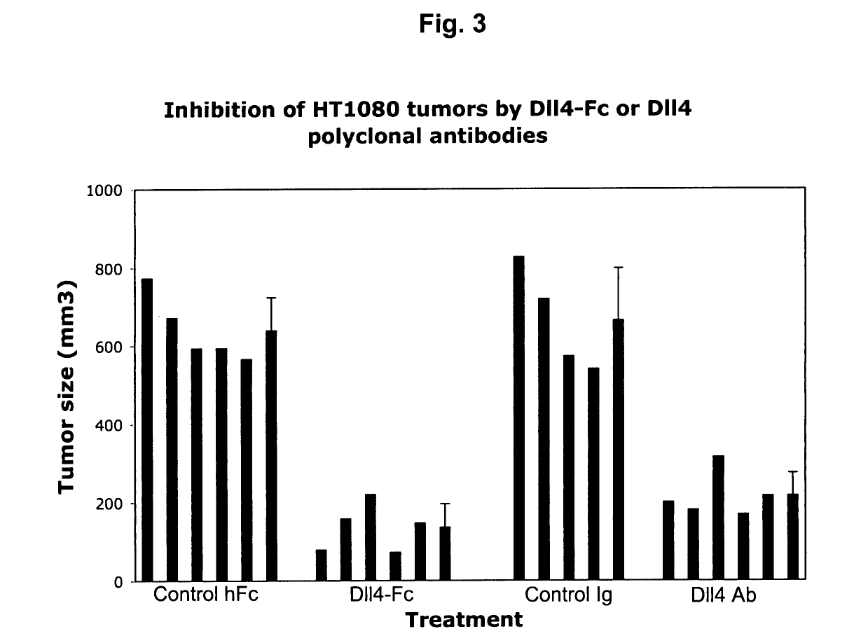

Experiments were conducted in which HT1080 tumors were implanted into mice on day 0 as described above. When the tumors reached 100 mm³ (approximately at day 15), mice were treated three times per week with DII4-Fc alone (25 mg/kg), control antibody (rabbit Ig), or anti-DII4 polyclonal antibodies depleted for binding to human Fc (10 mg/kg). Results show tumor size in each treatment group ±S.D. (FIG. 3) DII4 antibodies were highly effective against HT1080 tumor growth and had an effectiveness similar to that seen with DII4-Fc. These results show that a specific blocker of DII4 is a potent anti-tumor agent.

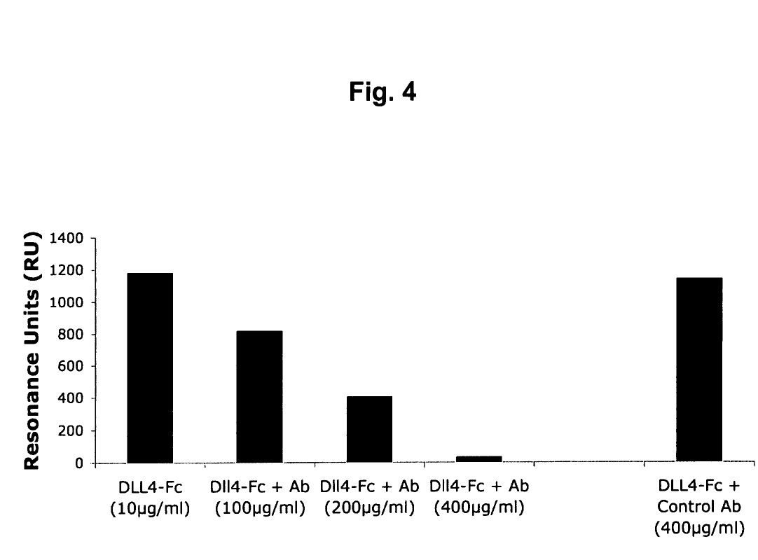

Surface plasmon resonance (BiaCore®) assays were performed confirming the DII4 antibodies were capable of blocking DII4 binding to Notch receptor. Notch 1 was coated on the chip surface and DII4-Fc was incubated with increasing amounts of rabbit polyclonal anti-DII4 antibody (described above). The results in FIG. 4 show that increasing amount of DII4 antibody increasingly blocked DII4-Fc binding to Notch1 (control=DII4-Fc+non-specific rabbit polyclonal antibody).

What is claimed is:

1. A therapeutic method for inhibiting tumor development or growth, comprising administering an agent capable of inhibiting DII4 activity to a subject in need thereof.

2. The therapeutic method of claim 1, wherein the DII4 antagonist is an antibody or antibody fragment capable of blocking the binding of DII4 to a Notch receptor.

3. The therapeutic method of claim 2, wherein the 0114 antibody or antibody fragment is polyclonal or monoclonal.

4. The therapeutic method of claim 3, wherein the antibody or antibody fragment is humanized, chimeric, or fully human antibody or antibody fragment.

5. The therapeutic method of claim 4, wherein the antibody fragment is a single chain antibody, an Fab, or an F(ab′)₂.

6. The therapeutic method of claim 1, wherein the DII4 antagonist is a fragment of DII4 fused to a multimerizing component.

7. The therapeutic method of claim 1, further comprising administering a second therapeutic agent which is a vascular endothelial growth factor (VEGF) inhibitor.

8. The method of claim 7, wherein the VEGF inhibitor is anti-VEGF antibody or a VEGF trap.

9. The method of claim 8, wherein the VEGF trap is SEQ ID NO:19.
